(19) United States
(12) Patent Application Publication
(10) Pub. No.: US 2023/0087844 A1
(43) Pub. Date: Mar. 23, 2023

(54) USE OF N2-QUINCLINE OR ISOQUINOLINE SUBSTITUTED PURINE DERIVATIVES IN CANCER TREATMENT

(71) Applicant: SHANGHAI HUAYU BIOTECHNOLOGY CO., LTD., Shanghai (CN)

(72) Inventor: Zhanggui WU, Brookline, MA (US)

(21) Appl. No.: 17/788,338

(22) PCT Filed: Jan. 11, 2021
(86) PCT No.: PCT/US2021/012907

§ 371 Date: Jun. 23, 2022

Related U.S. Application Data
(60) Provisional application No. 62/960,150, filed on Jan. 13, 2020.

(51) Int. Cl. A61K 31/52; A61P 35/00

(52) U.S. Cl. CPC A61K 31/52; A61P 35/00

(57) ABSTRACT

Disclosed is the use of the use of N²-quinoline or isoquinoline substituted purine derivatives in leukemia treatment, and the use of such purine derivatives in combination with a poly(ADP)-ribose polymerase (PARP) inhibitor and/or a chemotherapeutic agent in cancer treatment.

### RELATED APPLICATIONS AND INCORPORATION BY REFERENCE

This application claims priority to U.S. provisional application No. 62/960,150 filed on Jan. 13, 2020.

The foregoing application, and all documents cited therein or during its prosecution (“appln cited documents”) and all documents cited or referenced herein (including without limitation all literature documents, patents, published patent applications cited herein) (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference. Any Genbank sequences mentioned in this disclosure are incorporated by reference with the Genbank sequence to be that of the earliest effective filing date of this disclosure.

### TECHNICAL FIELD

The present disclosure relates to the use of N²-quinoline or isoquinoline substituted purine derivatives in leukemia treatment, and the use of such purine derivatives in combination with a chemotherapeutic agent and/or a poly(ADP)-ribose polymerase (PARP) inhibitor in cancer treatment.

### BACKGROUND OF THE INVENTION

Cancer is a group of diseases involving abnormal cell growth, with the potential to spread to other parts of body, causing about 600,000 people die in the United States in 2019.

There are more than 100 types of cancer, most of the carcinogenesis mechanisms remain poorly understood. Among these, leukemia is one that affects both children and adults, and is the most common cancer found in children. Generally speaking, leukemia is a kind of blood cancer that usually begins in bone marrow and results in a high level of abnormal blood cells that do not function properly, e.g., abnormal white blood cells that no longer protect the body from invasion by bacteria, viruses and fungi. With the disease development, symptoms such as fatigue, bleeding, bruising, and fever arise. Leukemia can be either acute or chromic, depending on how quickly the disease develops, and can also be grouped based on the type of blood cell as affected, i.e., the lymphoid cells or myeloid cells. Acute lymphocytic leukemia (ALL) is the most common type in young children, the chronic lymphocytic leukemia (CLL) most affects adults over 55, the chronic myeloid leukemia (CML) occur mainly in adults, while the acute myeloid leukemia (AML) occur in both adults and children.

The current options for cancer treatment include surgery, radiation therapy, chemotherapy, hormone therapy, and immunotherapy. The traditional radiation therapy and chemotherapy were and still are first-line treatments for various types of cancers, such as leukemia, due to efficient destruction of cancer cells. However, these two commonly adopted therapies, especially chemotherapy, are usually quite toxic to patients as they usually non-selectively destroy healthy cells and cause depression of the immune system. Therefore, there is always a need for a chemotherapeutic agent with a higher therapeutic efficacy but lower toxicity.

Another trend in cancer treatment is the combination therapy. For example, the radiation therapy or chemotherapy can be combined with immunotherapy or targeted therapy. Alternatively, two chemotherapeutic agents targeting different cancer-inducing or sustaining molecules or pathways may be used in combination. Such combination therapies may result in increased chance of killing cancer cells, minimized drug resistance and lower single drug dose. However, not all therapies or therapeutic agents can be combined and even few combination treatments work in a synergistic manner, as, e.g., one therapeutic agent may change a secondary agent's conformation and thus disable its anti-tumor activity. Alternatively, one therapeutic agent may inhibit the metabolism of a secondary agent in human body, leading to the buildup of toxicity. For example, the combination of panobinostat and carfilzomib caused treatment-related heart failure (2%), and treatment-related death rose by 2% in patients with relapsed/refractory multiple myeloma (Berdeja JG et al., (2015) Haematologica 100(5):670-676). Full investigation is thus needed on the interaction between two or more anti-tumor agents in a combination regimen before coming to a conclusion that whether the combination regimen is proper or not, or whether a maximum efficacy may be achieved with minimal toxicity.

The N²-quinoline or isoquinoline substituted purine derivatives disclosed in US2006/0293274A have been found to inhibit the activity of several kinases, such as phosphoinositide 3-kinase (PI3K), protein kinase B (PKB, also known as AKT) and platelet-derived growth factor receptor (PDGFR), and proved to be good anti-cancer candidates. Studies are needed to explore the combination of these compounds with one or more additional therapeutic agents, such as a chemotherapeutic agent or a therapeutic agent targeting PARP, a family of proteins that detect and initiate immediate cellular responses to single-strand DNA breaks (SSB), in cancer treatment. The addition of the additional agent(s) should improve the overall anti-tumor effect and not increase the toxicity.

Citation or identification of any document in this application is not an admission that such document is available as prior art to the present disclosure.

### SUMMARY OF THE INVENTION

The present inventor has surprisingly found that the compounds disclosed in US2006/0293274A1 are highly efficacious in treating leukemia especially acute myeloid leukemia (AML), with the tumor growth inhibition rate being much higher in leukemia model than other cancer models. Further, the present inventor has surprisingly found that these compounds work synergistically with a chemotherapeutic agent and/or a PARP inhibitor in cancer treatment.

Therefore, in one aspect, the present disclosure discloses a method for treating leukemia in a subject in need thereof, comprising administering a therapeutically effective amount of a compound of formula (I), or a pharmaceutically acceptable salt or solvent thereof,

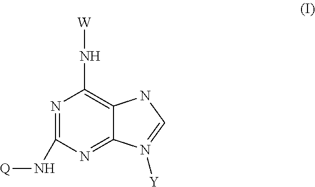

wherein Q is hydrogen, or one of the following:

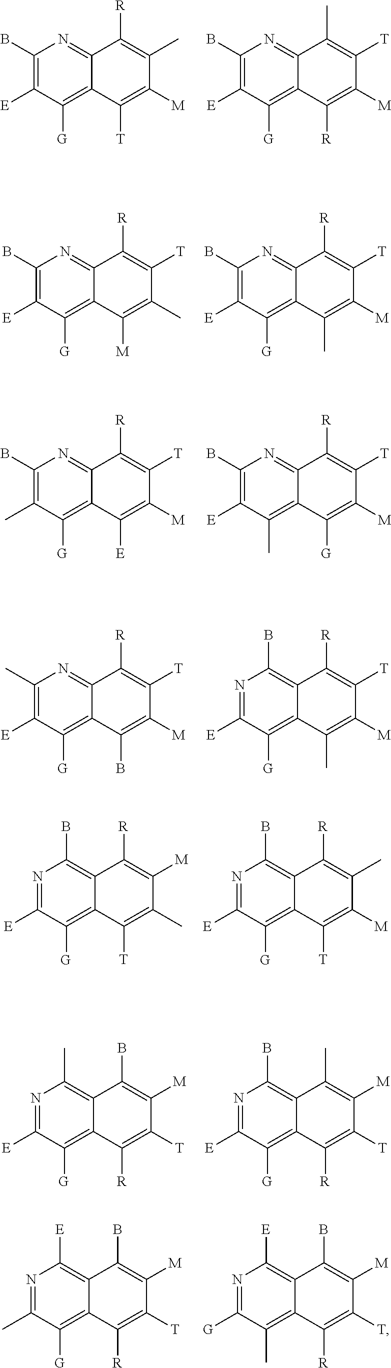

wherein B, E, G, R, T and M are independently hydrogen, an C₁₋₆ alkyl, an C₃₋₆ cycloalkyl, a halogen, a cyano, or an amino group,
W is hydrogen, an optionally substituted C₁₋₆ alkyl, an optionally substituted C₃₋₆ cycloalkyl, or an optionally substituted C₁₋₆ haloalkyl, and
Y is hydrogen, or a saccharide.

W in formula (I) is preferably one of the following:

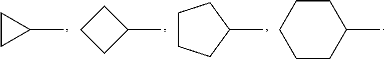

Q in formula (I) is preferably one of the following:

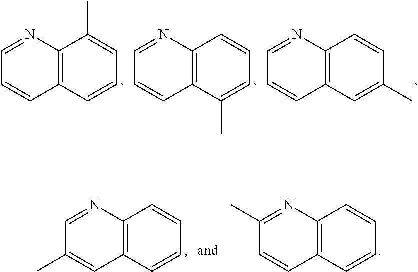

When Y in formula (I) is a saccharide, it is pharmaceutically acceptable and preferably one of the following:

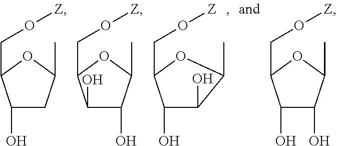

wherein Z is hydrogen or one of the following:

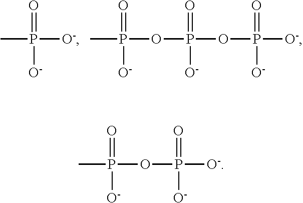

In one embodiment, W is preferably

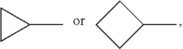

and Q is one of the following:

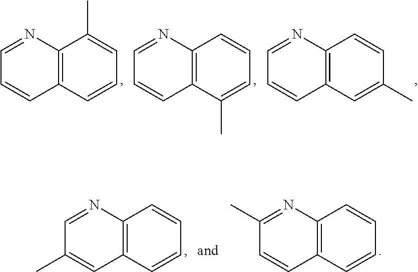

Q is preferably

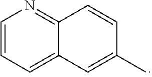

The compound of formula (I) may be selected from the group consisting of:

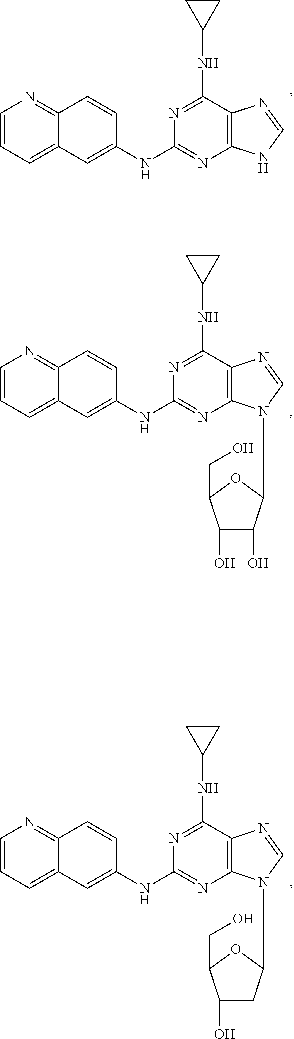 [SMILES: c1cnc2ccc(Nc3nc(NC4CC4)c4nc[nH]c4n3)cc2c1 ; OCC1OC(n2cnc3c(NC4CC4)nc(Nc4ccc5ncccc5c4)nc32)C(O)C1O ; OCC1OC(n2cnc3c(NC4CC4)nc(Nc4ccc5ncccc5c4)nc32)CC1O]

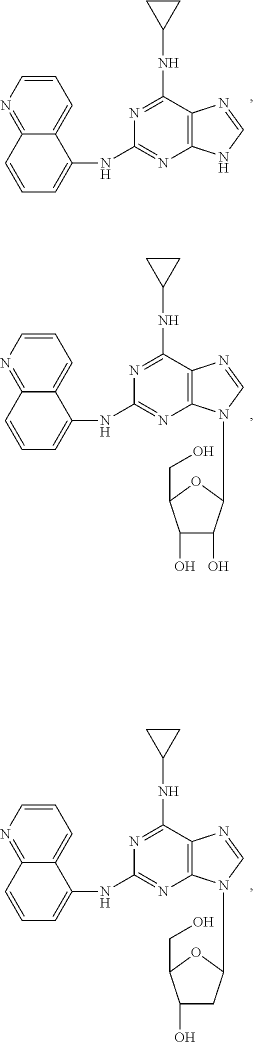 [SMILES: c1cc(Nc2nc(NC3CC3)c3nc[nH]c3n2)c2cccnc2c1 ; OCC1OC(n2cnc3c(NC4CC4)nc(Nc4cccc5ncccc45)nc32)C(O)C1O ; OCC1OC(n2cnc3c(NC4CC4)nc(Nc4cccc5ncccc45)nc32)CC1O]

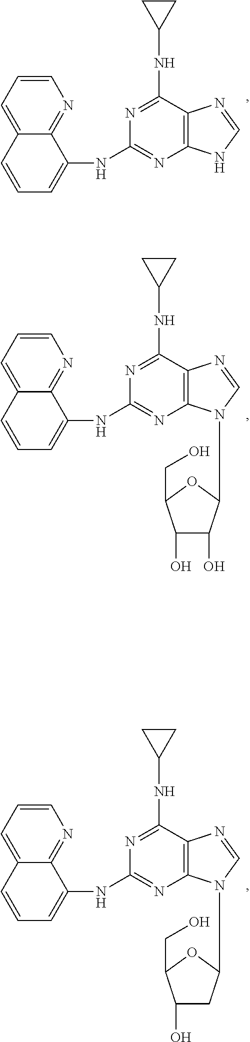 [SMILES: c1cnc2c(Nc3nc(NC4CC4)c4nc[nH]c4n3)cccc2c1 ; OCC1OC(n2cnc3c(NC4CC4)nc(Nc4cccc5cccnc45)nc32)C(O)C1O ; OCC1OC(n2cnc3c(NC4CC4)nc(Nc4cccc5cccnc45)nc32)CC1O]

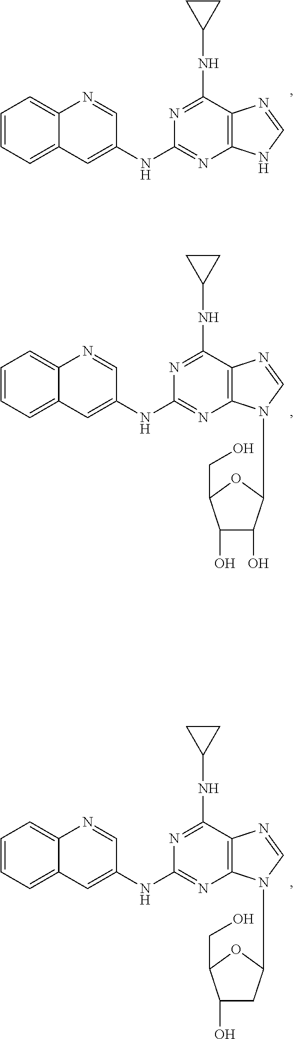 [SMILES: c1ccc2ncc(Nc3nc(NC4CC4)c4nc[nH]c4n3)cc2c1 ; OCC1OC(n2cnc3c(NC4CC4)nc(Nc4cnc5ccccc5c4)nc32)C(O)C1O ; OCC1OC(n2cnc3c(NC4CC4)nc(Nc4cnc5ccccc5c4)nc32)CC1O]

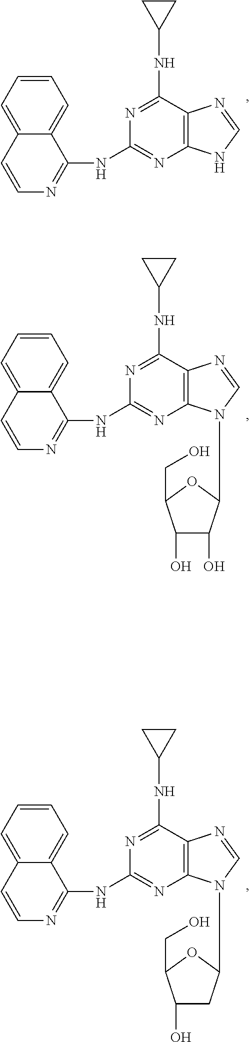 [SMILES: c1ccc2c(Nc3nc(NC4CC4)c4nc[nH]c4n3)nccc2c1 ; OCC1OC(n2cnc3c(NC4CC4)nc(Nc4nccc5ccccc45)nc32)C(O)C1O ; OCC1OC(n2cnc3c(NC4CC4)nc(Nc4nccc5ccccc45)nc32)CC1O]

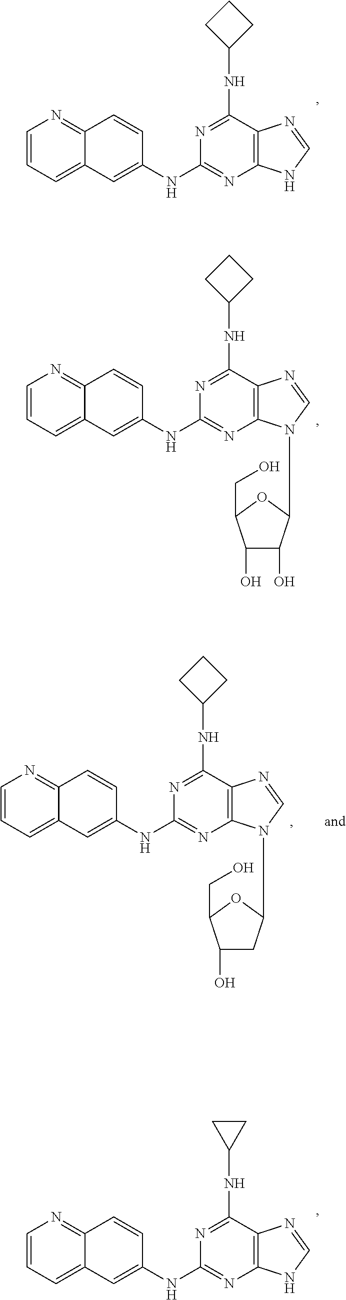 [SMILES: c1cnc2ccc(Nc3nc(NC4CCC4)c4nc[nH]c4n3)cc2c1 ; OCC1OC(n2cnc3c(NC4CCC4)nc(Nc4ccc5ncccc5c4)nc32)C(O)C1O ; OCC1OC(n2cnc3c(NC4CCC4)nc(Nc4ccc5ncccc5c4)nc32)CC1O ; c1cnc2ccc(Nc3nc(NC4CC4)c4nc[nH]c4n3)cc2c1]

In certain embodiments, the compound is

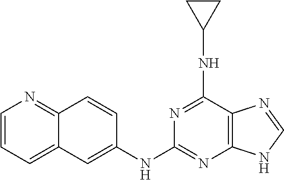 [SMILES: c1cnc2ccc(Nc3nc(NC4CC4)c4nc[nH]c4n3)cc2c1]

with an extremely high activity, also referred to as Compound A hereinafter.

Leukemia may be acute leukemia or chronic leukemia. The acute leukemia may be acute lymphocytic leukemia (ALL) or acute myeloid leukemia (AML). The chronic leukemia may be chronic lymphocytic leukemia (CLL) or chronic myeloid leukemia (CML). The acute lymphocytic leukemia may be B-cell ALL or T-cell ALL. The acute myeloid leukemia may be acute monocytic leukemia or acute myeloblastic leukemia.

The compound may be administered every day or every other day at a daily dose at 10-500 mg/kg. In one embodiment, the compound is administered every day at a daily dose of 0.1-200 mg/kg, preferably 0.2-100 mg/kg, and most preferably 0.3-50 mg/kg.

In another aspect, the present disclosure discloses a method for treating a cancer in a subject in need thereof, comprising administering a therapeutically effective amount of a compound of formula (I), or a pharmaceutically acceptable salt or solvent thereof, as described above, in combination with a chemotherapeutic agent and/or a therapeutic agent targeting PARP in cancer treatment.

The chemotherapeutic agent suitable for the present invention may be cisplatin, pemetrexed, gemcitabine, cytarabine, hydroxycarbamide, temozolomide, irinotecan, cyclophosphamide, mitoxantrone, etoposide, folinic acid, fludarabine, fluorouracil, or a combination thereof. In one embodiment, the chemotherapeutic agent is gemcitabine. In one embodiment, the chemotherapeutic agent is daunorubicin.

The therapeutic agent targeting PARP may be a PARP inhibitor, such as a molecule having inhibitory effect on PARP activity. The PARP inhibitor suitable for the present invention may be selected from the group consisting of olaparib, rucaparib, niraparib, and talazoparib. In one embodiment, the PARP inhibitor is olaparib. In one embodiment, the PARP inhibitor is rucaparib.

The cancer may be a solid cancer selected from the group consisting of lung, prostate, ovarian, brain, breast, skin, bladder, colon, gastrointestinal, head and neck, gastric, pancreas, neurologic, renal, and liver cancer. In certain embodiments, the cancer is ovarian, prostate, gastric or breast cancer. In one embodiment, the cancer is BRCA-mutant. In certain embodiments, the cancer is lung cancer. In one embodiment, the cancer is small cell lung cancer. In certain embodiments, the cancer is colon cancer.

The cancer may be a hematological cancer selected from the group consisting of lymphocytic leukemia, myeloid leukemia, non-Hodgkin lymphoma, and Hodgkin lymphoma. In certain embodiments, the cancer is myeloid leukemia. In one embodiment, the cancer is acute myeloid leukemia (AML).

The racemates, pure stereoisomers, in particular enantiomers or diastereomers, and the mixtures of stereoisomers in any mixing ratio of the compounds described above are also used in the cancer treatment method of the disclosure.

The present disclosure also discloses the use of the compound of formula (I) alone in leukemia treatment, and the use of the compound of formula (I) in cancer treatment in combination with a chemotherapeutic agent and/or a PARP inhibitor.

Other features and advantages of the instant disclosure, literally described and their equivalents understood by those ordinarily skilled in the art, will be apparent from the following drawings, detailed description and examples, as well as claims, which should not be construed as limiting. The contents of all publications, references, Genbank entries, patents and published patent applications cited throughout this application are expressly incorporated herein by reference.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 is a column graph showing the death rates of H69 cells treated with Compound A, olaparib, and Compound A + olaparib, respectively.

FIG. 2 is a column graph showing the death rates of H526 cells treated with Compound A, olaparib, and Compound A + olaparib, respectively.

FIG. 3 a column graph showing the death rates of H446 cells treated with Compound A, olaparib, and Compound A + olaparib, respectively.

FIG. 4 is a column graph showing the death rates of H69 cells treated with Compound A, rucaparib, and Compound A + rucaparib, respectively.

FIG. 5 is a column graph showing the death rates of H526 cells treated with Compound A, rucaparib, and Compound A + rucaparib, respectively.

FIG. 6 is a column graph showing the death rates of H446 cells treated with Compound A, rucaparib, and Compound A + rucaparib, respectively.

### DETAILED DESCRIPTION OF THE INVENTION

Before particular embodiments of the present disclosure are disclosed and described, it is to be understood that this disclosure is not limited to the particular process and materials disclosed herein as such may vary to some degree. It is also to be understood that the terminology used herein is used for the purpose of describing particular embodiments only and is not intended to be limiting, as the scope of the present disclosure will be defined only by the appended claims and equivalents thereof.

The singular forms “a,” “an,” and “the” include plural referents unless the context clearly dictates otherwise.

Unless otherwise indicated, any heteroatom with unsatisfied valences is assumed to have hydrogen atoms sufficient to satisfy the valences.

Throughout the specification and the appended claims, a given chemical formula or name shall encompass all stereo and optical isomers and racemates thereof where such isomers exist. Unless otherwise indicated, all chiral (enantiomeric and diastereomeric) and racemic forms are within the scope of the disclosure. Many geometric isomers of C═C double bonds, C═N double bonds, ring systems, and the like can also be present in the compounds, and all such stable isomers are contemplated in the present disclosure. Cis- and trans- (or E- and Z-) geometric isomers of the compounds of the present disclosure are described and may be isolated as a mixture of isomers or as separated isomeric forms. The present compounds can be isolated in optically active or racemic forms. Optically active forms may be prepared by resolution of racemic forms or by synthesis from optically active starting materials. All processes used to prepare compounds of the present disclosure and intermediates made therein are considered to be part of the present disclosure. When enantiomeric or diastereomeric products are prepared, they may be separated by conventional methods, for example, by chromatography or fractional crystallization. Depending on the process conditions the end products of the present disclosure are obtained either in free (neutral) or salt form. Both the free form and the salts of these end products are within the scope of the disclosure. If so desired, one form of a compound may be converted into another form. A free base or acid may be converted into a salt; a salt may be converted into the free compound or another salt; a mixture of isomeric compounds of the present disclosure may be separated into the individual isomers. Compounds of the present disclosure, free form and salts thereof, may exist in multiple tautomeric forms, in which hydrogen atoms are transposed to other parts of the molecules and the chemical bonds between the atoms of the molecules are consequently rearranged. It should be understood that all tautomeric forms, insofar as they may exist, are included within the disclosure.

As referred to herein, the term “substituted” means that at least one hydrogen atom is replaced with a non-hydrogen group, provided that normal valencies are maintained and that the substitution results in a stable compound.

When a substituent is noted as “optionally substituted”, the substituents are selected from, for example, substituents such as alkyl, cycloalkyl, aryl, heterocyclo, halo, hydroxy, alkoxy, oxo, alkanoyl, aryloxy, alkanoyloxy, amino, alkylamino, arylamino, arylalkylamino, disubstituted amines in which the 2 amino substituents are selected from alkyl, aryl or arylalkyl; alkanoylamino, aroylamino, aralkanoylamino, substituted alkanoylamino, substituted arylamino, substituted aralkanoylamino, thiol, alkylthio, arylthio, arylalkylthio, alkylthiono, arylthiono, arylalkylthiono, alkylsulfonyl, arylsulfonyl, arylalkylsulfonyl, sulfonamido, e.g. —SO₂NH₂, substituted sulfonamido, nitro, cyano, carboxy, carbamyl, e.g. —CONH₂, substituted carbamyl e.g. —CONHalkyl, —CONHaryl, —CONHarylalkyl or cases where there are two substituents on the nitrogen selected from alkyl, aryl or arylalkyl; alkoxycarbonyl, aryl, substituted aryl, guanidino, heterocyclyl, e.g., indolyl, imidazolyl, furyl, thienyl, thiazolyl, pyrrolidyl, pyridyl, pyrimidyl, pyrrolidinyl, piperidinyl, morpholinyl, piperazinyl, homopiperazinyl and the like, and substituted heterocyclyl, unless otherwise defined.

As used herein, the term “alkyl” or “alkylene” is intended to include both branched and straight-chain saturated aliphatic hydrocarbon groups having the specified number of carbon atoms. For example, “C₁-C₆ alkyl” denotes alkyl having 1 to 6 carbon atoms. Exemplary alkyl groups include, but are not limited to, methyl (Me), ethyl (Et), propyl (e.g., n-propyl and isopropyl), butyl (e.g., n-butyl, isobutyl, t-butyl), and pentyl (e.g., n-pentyl, isopentyl, neopentyl).

The term “alkenyl” denotes a straight- or branch-chained hydrocarbon radical containing one or more double bonds and typically from 2 to 20 carbon atoms in length. For example, “C₂-C₈ alkenyl” contains from two to eight carbon atoms. Alkenyl groups include, but are not limited to, for example, ethenyl, propenyl, butenyl, 1-methyl-2-buten-1-yl, heptenyl, octenyl and the like.

The term “alkynyl” denotes a straight- or branch-chained hydrocarbon radical containing one or more triple bonds and typically from 2 to 20 carbon atoms in length. For example, “C₂-C₈ alkenyl” contains from two to eight carbon atoms. Representative alkynyl groups include, but are not limited to, for example, ethynyl, 1-propynyl, 1- butynyl, heptynyl, octynyl and the like.

The term “alkoxy” or “alkyloxy” refers to an —O-alkyl group. “C₁-C₆ alkoxy” (or alkyloxy), is intended to include C₁, C₂, C₃, C₄, C₅, and C alkoxy groups. Exemplary alkoxy groups include, but are not limited to, methoxy, ethoxy, propoxy (e.g., n-propoxy and isopropoxy), and t-butoxy. Similarly, “alkylthio” or “thioalkoxy” represents an alkyl group as defined above with the indicated number of carbon atoms attached through a sulphur bridge; for example methyl-S— and ethyl-S—.

The term “aryl”, either alone or as part of a larger moiety such as “aralkyl”, “aralkoxy”, or “aryloxyalkyl”, refers to monocyclic, bicyclic and tricyclic ring systems having a total of five to 15 ring members, wherein at least one ring in the system is aromatic and wherein each ring in the system contains three to seven ring members. In certain embodiments of the disclosure, “aryl” refers to an aromatic ring system which includes, but not limited to phenyl, biphenyl, indanyl, 1-naphthyl, 2-naphthyl and terahydronaphthyl. The term “aralkyl” or “arylalkyl” refers to an alkyl residue attached to an aryl ring. Non-limiting examples include benzyl, phenethyl and the like. The fused aryls may be connected to another group either at a suitable position on the cycloalkyl ring or the aromatic ring. For example:

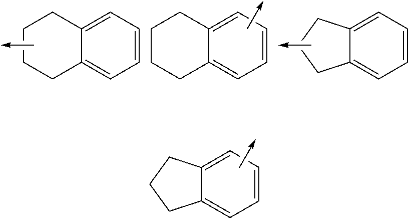

Arrowed lines drawn from the ring system indicate that the bond may be attached to any of the suitable ring atoms.

The term “cycloalkyl” refers to cyclized alkyl groups. C₃-C₆ cycloalkyl is intended to include C₃, C₄, C₅, and C₆ cycloalkyl groups. Exemplary cycloalkyl groups include, but are not limited to, cyclopropyl, cyclobutyl, cyclopentyl, cyclohexyl, and norbornyl. Branched cycloalkyl groups such as 1-methylcyclopropyl and 2-methylcyclopropyl are included in the definition of “cycloalkyl”. The term “cycloalkenyl” refers to cyclized alkenyl groups. C₄₋₆ cycloalkenyl is intended to include C₄, C₅, and C₆ cycloalkenyl groups. Exemplary cycloalkenyl groups include, but are not limited to, cyclobutenyl, cyclopentenyl, and cyclohexenyl.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C—, ClCH₂—, CF₃CH₂— and CF₃CCl₂—. The terms “haloalkoxy” and “haloalkylthio” and the like, are defined analogously to the term “haloalkyl”. Examples of “haloalkoxy” include CF₃O—, CCl₃CH₂O—, HCF₂CH₂CH₂O— and CF₃CH₂O—.

As used herein, the term “heterocycle,” “heterocyclyl,” or “heterocyclic group” is intended to mean a stable 4-, 5-, or 6-membered monocyclic that is saturated, partially unsaturated, or fully unsaturated, and that contains carbon atoms and 1, 2, 3 or 4 nitrogen, oxygen or other non-carbon atoms.

In cases wherein there are nitrogen atoms (e.g., amines) on compounds of the present disclosure, these may be converted to N-oxides by treatment with an oxidizing agent (e.g., mCPBA and/or hydrogen peroxides) to afford other compounds of this disclosure. Thus, shown and claimed nitrogen atoms are considered to cover both the shown nitrogen and its N-oxide (N- >0) derivative.

When any variable occurs more than one time in any constituent or formula for a compound, its definition at each occurrence is independent of its definition at every other occurrence. Thus, for example, if a group is shown to be substituted with 0-3 R, then said group may optionally be substituted with up to three R groups, and at each occurrence R is selected independently from the definition of R. Also, combinations of substituents and/or variables are permissible only if such combinations result in stable compounds.

When a bond to a substituent is shown to cross a bond connecting two atoms in a ring, then such substituent may be bonded to any atom on the ring. When a substituent is listed without indicating the atom in which such substituent is bonded to the rest of the compound of a given formula, then such substituent may be bonded via any atom in such substituent. Combinations of substituents and/or variables are permissible only if such combinations result in stable compounds.

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, and/or other problem or complication, commensurate with a reasonable benefit/risk ratio. As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like. A “pharmaceutically acceptable solvate” refers to a multicomponent crystalline solid molecular adduct containing the hose molecule (e.g., the compound of formula (I)) and guest solvent molecule(s) incorporated in the crystal lattice structure. When the solvent is water, the solvate is called hydrate. A “pharmaceutically acceptable carrier” refers to media generally accepted in the art for the delivery of biologically active agents to animals, in particular, mammals, including, i.e., adjuvant, excipient or vehicle, such as diluents, preserving agents, fillers, flow regulating agents, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington: The Science and Practice of Pharmacy, 22nd Edition, Allen, L. V. Jr., Ed.; Pharmaceutical Press, London, UK (2012), the disclosure of which is hereby incorporated by reference.

As used herein, the term “effective amount” means that amount of a drug or pharmaceutical agent, i.e., a compound of the disclosure, that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. An effective amount can be administered in one or more administrations, applications or dosages and is not intended to be limited to a particular formulation or administration route. The term also includes within its scope amounts effective to enhance normal physiological function

As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “parenteral” includes subcutaneous, intradermal, intravenous, intramuscular, intraarticular, intraarterial, synovial, intrasternal, intracranial, intramuscular or infusion.

The term “subject” includes any human or nonhuman animal. The term “nonhuman animal” includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses.

The term “IC₅₀” or “half maximal inhibitory concentration” refers to the concentration of an inhibitor where the response is reduced by half.

The term “lethal dose” or “LD”, in toxicology, is an indication of the lethal toxicity of a given substance such as the compound of formula (I) in the present disclosure. The median lethal dose, abbreviated as LD₅₀, of the compound refers to the dose required to kill half the members of a tested population after a specified test duration. The term “median toxic dose” or “TD₅₀” of a drug refers to a dose at which toxicity occurs in 50% of cases. The term “efficacious dose” or “ED” refers to a dose of a drug such as the compound of formula (I) in the present disclosure that produces a biological response. The median efficacious dose, abbreviated as ED₅₀, is a dose that produces a quantal effect in 50% of the population that takes the drug. The therapeutic index or therapeutic ratio can be calculated as the ratio of TD50 to ED50, or LD50 to ED50, which is a quantitative measurement of the relative safety of a drug.

### Compound of Formula (I)

The compounds of formula (I) have been disclosed in US2006/0293274A1, having low toxicity and high anti-tumor activity. The synthesis scheme and the function tests of these compounds have also been disclosed in that patent application. An exemplary compound is

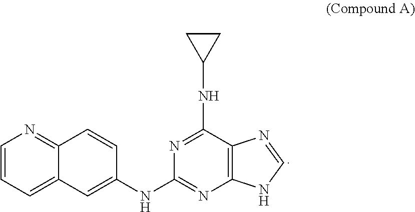 [SMILES: c1cnc2ccc(Nc3nc(NC4CC4)c4nc[nH]c4n3)cc2c1]

The inventor of the disclosure has surprisingly found that these compounds are particularly effective in the treatment of leukemia as compared to other cancers. Without bound to the theory, the inventor believes that such obviously better inhibition effect on leukemia is associated with the compounds' inhibitory effect on phosphoinositide 3-kinase (PI3K) δ isoform and FLT3.

In particular, the compounds of formula (I) are capable of inhibiting several kinases' activities, such as PI3K (including α, β, δ and γ isoforms), Protein kinase B (PKB, also known as AKT) and Platelet-derived growth factor receptor (PDGFR). The present inventor tested the inhibitory effect of Compound A on the four PI3K isoforms, using the method as described in CN104513254A, and found that the EC₅₀ with respect to δ isoform inhibition was about 90 nM, which was much lower than those for the other three isoforms at the μM level. The δ and γ isoforms are mainly expressed in leukocytes (Saudemont et al., (2009) Proc. Nat'l. Acad. Sci. 2009106: 5795-5800; Ali et al., (2014) Nature 510: 407-411), and thus the compound selectively against the δ isoform may be more active and efficacious in leukemia treatment.

The inventor further tested the inhibitory effect of Compound A on kinase insert domain receptor (KDR, also referred to as VEGFR-2), PDGFR, AKT and fms-like tyrosine kinase 3 (FLT3). The results showed that this compound inhibited the phosphorylation of KDR, PDGFR, AKT and FLT3 with EC₅₀ at about 1.0 μM, 1.0 μM, 0.1 μM and 49.0 nM, respectively, suggesting its high inhibition activity on FLT3. FLT3 is a class III receptor tyrosine kinase and plays an important role in hematopoiesis and lymphocyte development. The abnormal FLT3 activation was found to be closely associated with the development of several tumors such as AML (Griffith et al., (2004)Mol Cell. 13:169-78). Wang et al found over 60% of 82 leukemia cell lines were FLT3 positive (Wang Y et al., (2006) Journal of Experimental Hematology 14(3): 446-449). FLT3-internal tandem duplication (ITD) is the most commonly seen mutation in AML patients, with about 17-34% of AML patients carrying such a mutation (Jia H et al., (2018) Medical Laboratory Science and Clinics 19(5): 39). FTL3 inhibitors such as quizartinib, sorafenib and gilteritinib are clinically used in acute myeloblastic leukemia treatment and resulted in good clinical outcomes (Qi L et al., (2014) Chinese Journal of Cancer Biotherapy 21(1): 20-24). Thus, the compounds of the disclosure with high inhibitory effect on FLT3 function may be good candidates for leukemia treatment.

### Chemotherapeutic Agent

The chemotherapeutic agent herein refers to a powerful chemical that kills fast-growing cells in the body. Such an agent is usually used to treat cancers, as cancer cells grow and divide faster than other cells.

Chemotherapeutic agents for cancer treatment include, but not limited to, cisplatin, pemetrexed, gemcitabine, cytarabine, hydroxycarbamide, temozolomide, irinotecan, cyclophosphamide, mitoxantrone, etoposide, folinic acid, fludarabine, and fluorouracil.

Gemcitabine, a chemotherapy medication used in treatment of a number of types of cancers, is a ribonucleotide reductase inhibitor that leads to dNTP depletion and fork stalling, blocking the formation of new DNAs. It was first approved in 1995 for medical use, and is now used as a first-line treatment alone for pancreatic cancer, and in combination with cisplatin for advanced or metastatic bladder cancer and advanced or metastatic non-small cell lung cancer. It is also used as a second-line treatment in combination with carboplatin for ovarian cancer and in combination with paclitaxel for breast cancer that is metastatic or cannot be surgically removed. Gemcitabine use may cause side effects such as bone marrow suppression, liver and kidney problems, nausea, fever, and hair loss.

Cisplatin is another chemotherapy medication commonly used in treatment of a number of cancers. It was discovered in 1845 and put into medical use in 1978. It works by binding to DNA and thus inhibiting DNA replication, and is used to treat sarcomas, SCLC, ovarian cancer and etc.

### PARP Inhibitor

PARP is a family of proteins that catalyze the transfer of ADP-ribose to target proteins. It plays an important role in several cellular processes, including transcription, replication, recombination and DNA repair, among which DNA repair is of particular interest where PARP detects and initiates immediate cellular responses to single-strand DNA breaks (SSB).

Certain tumors defective in homologous recombination repair (HRR) mechanisms, e.g., BRCA-mutant cancers, may rely on PARP-mediated DNA repair for survival, and thus sensitive to PARP inhibition. In particular, PARP inhibitors impair SSB repair, leading to double-strand DNA breaks (DSB). When such DSBs cannot be efficiently repaired by HRR, cancer cell death may occur (Martin SA, et al., (2008) Curr Opin Genet Dev 18:80-86). The PARP inhibitors are usually used to increase tumor sensitivity to DNA-damaging agents.

PI3K blockade was reported to downregulate BRCA½ expression and leads to homologous recombination deficiency, and is effective in inhibiting breast and ovarian cancers when combined with PARP inhibition (R. Condorelli et al., (2017) Annals of Oncology 28(6): 1167-1168). The dual-blockade of PI3K and PARP was also found to be promising in treatment of certain prostate and gastric cancers (Yang L et al., (2018) Oncol Rep. 40(1): 479-487; González-Billalabeitia, E. et al., (2014) Cancer Discov. 4: 896-904).

### Combination Therapy

The compound of formula (I) may be used in combination with a chemotherapeutic agent and/or a PARP inhibitor, to gain a better anti-cancer effect and/or a lower toxicity to human body.

The compound of formula (I), and the chemotherapeutic agent/the PARP inhibitor may be administered concurrently as a single composition in a pharmaceutically acceptable carrier, or concurrently as separate compositions. They can also be administered sequentially.

The combination therapy of the present disclosure may be used to treat a cancer, such as a solid cancer selected from the group consisting of lung, prostate, ovarian, brain, breast, skin, bladder, colon, gastrointestinal, head and neck, gastric, pancreas, neurologic, renal, and liver cancer, or a hematological cancer selected from the group consisting of lymphocytic leukemia, myeloid leukemia, non-Hodgkin lymphoma, and Hodgkin lymphoma.

The combination therapy of the present disclosure may be applied to animals, preferably mammals (e.g., domesticated animals, cats, dogs, mice, rats), and more preferably humans. Any method of administration may be used to deliver the compound of formula (I), and the chemotherapeutic agent/the PARP inhibitor to the subject in need thereof. In certain embodiments, the compound of formula (I) and the chemotherapeutic agent/the PARP inhibitor are administered orally. In other embodiments, the compound of formula I and the chemotherapeutic agent/the PARP inhibitor are administered parenterally.

One or more additional pharmaceutical agents or treatment methods such as, for example, other chemotherapeutics or other anti-cancer agents, immune enhancers, immunosuppressants, anti-tumor vaccines, and/or cytokine therapy (e.g., IL2 and GM-CSF) can be optionally used in combination with the combination therapy of the disclosure. The additional agents can be combined with the compound of formula (I) and the chemotherapeutic agent/the PARP inhibitor in a single dosage form, or these agents can be administered simultaneously or sequentially as separate dosage forms.

### Pharmaceutical Compositions and Dosing

The disclosure also provides pharmaceutically acceptable compositions which comprise a therapeutically effective amount of one or more compounds of Formula (I), formulated together with one or more pharmaceutically acceptable carriers (additives) and/or diluents, with or without (i) a therapeutically effective amount of the chemotherapeutic agent mentioned above, formulated together with one or more pharmaceutically acceptable carriers (additives) and/or diluents, (ii) a therapeutically effective amount of the PARP inhibitor mentioned above, formulated together with one or more pharmaceutically acceptable carriers (additives) and/or diluents, and/or (iii) optionally, one or more additional therapeutic agents described above if needed. The compounds of this disclosure, the chemotherapeutic agent, and/or the PARP inhibitor can be administered by any suitable means, for example, orally, as tablets, capsules (each of which includes sustained release or timed release formulations), pills, powders, granules, elixirs, tinctures, suspensions (including nanosuspensions, microsuspensions, spray-dried dispersions), syrups, and emulsions; sublingually; bucally; parenterally, such as by subcutaneous, intravenous, intramuscular, or intrasternal injection, or infusion techniques (e.g., as sterile injectable aqueous or non-aqueous solutions or suspensions); nasally, including administration to the nasal membranes, such as by inhalation spray; topically, such as in the form of a cream or ointment; or rectally such as in the form of suppositories. The pharmaceutical composition of the present disclosure can also be prepared as liposomes and nanoparticles.

The dosage regimen for the pharmaceutical compositions of the disclosure will, of course, vary depending upon known factors, such as the pharmacodynamic characteristics of the particular agents and the mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. By way of general guidance, the daily oral dosage of each active ingredient, when used for the indicated effects, will range between about 0.001 to about 5000 mg per day. Intravenously, the most preferred doses will range from about 0.01 to about 10 mg/kg/minute during a constant rate infusion. The pharmaceutical composition of this disclosure may be administered in a single daily dose, or the total daily dosage may be administered in divided doses of two, three, or four times daily.

The toxicity and efficacy of the pharmaceutical compositions, with or without the chemotherapeutic agent and/or the PARP inhibitor, can be tested in cell or animal based assays, and the assay data may serve as the basis for clinical dosing design. The pharmaceutic compositions of the disclosure with tolerated toxicity and high efficacy are preferable.

### EXAMPLES

### Example 1. Compound of Formula (I) Inhibited Tumor Cell Proliferation In Vitro

Compound A,

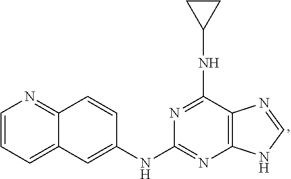 [SMILES: c1cnc2ccc(Nc3nc(NC4CC4)c4nc[nH]c4n3)cc2c1]

as an exemplary compound of formula (I), was tested for its inhibitory effect on tumor cell proliferation in a MTT assay. The tumor cell lines as tested were listed in Table 1 below.

TABLE 1
Tumor cell line listing

| Cell line | Source | Tumor type |
|---|---|---|
| A549 | ATCC | Human lung adenocarcinoma |
| NCI-H441 | ATCC | Human lung adenocarcinoma |
| NCI-H460 | ATCC | Human lung cancer |
| SGC-7901 | Cell Bank, Chinese | Human gastric cancer |
| | Academy of | |
| | Sciences, Shanghai | |
| SNU-5 | ATCC | Gastric cancer |
| Ls-174t | ATCC | Human colon adenocarcinoma |
| HT-29 | ATCC | Human colon cancer |
| U87 | ATCC | Human brain glioma |
| SK-BR-3 | ATCC | Human breast cancer |
| BT-474 | ATCC | Human breast cancer |
| MDA-MB-231 | ATCC | Human breast cancer |
| MCF-7 | ATCC | Human breast cancer |
| THP-1 | ATCC | Human acute monocytic leukemia |
| HL-60 | ATCC | Human acute promyelocytic leukemia |
| K562 | ATCC | Chronic myeloid leukemia |
| MV-4-11 | ATCC | Acute myeloid leukemia |
| RS 4;11 | ATCC | Acute lymphocytic leukemia |
| Reh | ATCC | Acute lymphocytic leukemia |

Briefly, cells at the log phase were collected, suspended in the cell culture medium at 4*10⁴/ml density, and plated in 96-well plates, 90 μl per well. The plates were kept in a 5% CO₂ and 37° C. incubator for 2 hours, and then added with 100 μg/ml, 10 μg/ml, 1 μg/ml, 0.1 μg/ml, 0.01 μg/ml or 0.001 μg/ml Compound A, 4 replicates for each cell at each compound concentration. DMSO was used in the positive control group, and another negative group was also set. After 72 hour incubation in the same incubator, 20 μl 5 mg/ml MTT reagent was added to each well. After another 4 hour incubation, each well was added with 100 μl SDS and further incubated for 12 hours. Absorbance at 570 nm was read in a reader, and IC₅₀ was calculated. In particular, the absorbance measurement was performed for three times, and the average IC₅₀ values were summarized in Table 2 below.

The data in Table 2 showed that Compound A inhibited the proliferation of all tumor cells above. In particular, this compound had a strong inhibitory effect on leukemia cell lines (IC₅₀ being about 1.6 μM or lower) while a relatively weaker effect on solid cancer cell lines (IC₅₀ being about 2.3 μM or higher).

TABLE 2
Inhibitory effect of Compound A on tumor cell proliferation

| Cell line | Tumor type | IC₅₀ (μM) |
|---|---|---|
| A549 | Lung cancer | 2.3 ± 0.8 |
| H441 | Lung cancer | 15.9 ± 9.5 |
| NCI-H460 | Lung cancer | 4.0 ± 0.4 |
| SGC-7901 | Gastric cancer | 3.8 ± 0.6 |
| SNU-5 | Gastric cancer | 12.1 ± 2.0 |
| Ls-174t | Colon cancer | 5.9 ± 0.9 |
| HT-29 | Colon cancer | 6.1 ± 0.2 |
| U87 | Brain glioma | 9.7 ± 7 |
| SK-BR-3 | Breast cancer | 16.5 ± 0.9 |
| BT-474 | Breast cancer | 4.4 ± 0.4 |
| MDA-MB-231 | Breast cancer | 3.5 ± 0.1 |
| MCF-7 | Breast cancer | 26.5 ± 7.7 |
| THP-1 | Acute monocytic leukemia | 1.6 ± 1.6 |
| HL-60 | Acute promyelocytic leukemia | 0.5 ± 0.3 |
| K562 | Chronic myeloid leukemia | 1.2 ± 0.1 |
| MV-4-11 | Acute myeloid leukemia | 0.3 ± 0.1 |
| RS 4;11 | Acute lymphocytic leukemia | 0.4 ± 0.1 |
| Reh | Acute lymphocytic leukemia | 0.8 ± 0.1 |

### Example 2. Compound of Formula (I) Inhibited Sarcoma Growth in Mice In Vivo

Kunming mice, 18-22 g, purchased from Shanghai SLAC Laboratory Animal Co., Ltd., were subcutaneously inoculated with S180 murine sarcoma cancer cells (from Shanghai Institute of Materia Medica, SIMM) at the right axilla. After the tumors grew to the size of 100-150 mm³, the tumor-bearing mice were randomly assigned to five groups on Day 1 (or D1), and administered with physiological saline, compound A at different doses/dosing routes, and cyclophosphamide (CTX, Hengrui Medicine), respectively. The detailed dosing regimen was listed in Table 3 below. On Day 8, the mice were weighed and sacrificed, and the tumors were collected and weighed.

Tumor growth inhibition (TGI) was calculated by the following formula. Tumor growth inhibition=(average tumor weight in vehicle group-average tumor weight in administration group)/average tumor weight in vehicle group×100%

The results were shown in Table 3. No mice died of tumor or drug administration. Both Compound A and CTX showed obvious anti-tumor effect, and oral administration of Compound A at the dose of 60 mg/kg from Day 1 to Day 4 achieved the best tumor growth inhibition effect.

TABLE 3
Dosing regimen and anti-tumor effect

| | | | | Body weight on | | | |
|---|---|---|---|---|---|---|---|
| | | | | Day 8 (without | | Tumor | |
| | Dose | | | tumor) (g) | | weight on | TGI |
| Group | (mg/kg) | N | Dosing Regimen | D1 | D8 | Day 8 (g) | (%) |
| Vehicle | n.a. | 16 | P.O., D1-4; I.P., D1-7 | 21.8 | 29.9 | 1.92 ± 0.41 | |
| Compound | 60 | 8 | P.O., D1-4 | 21.8 | 17.6 | 0.33 ± 0.13 | 82.8* |
| A | 80 | 8 | P.O., D1 &D4 | 21.9 | 23.4 | 1.12 ± 0.34 | 41.7* |
| | 80 | 8 | I.V., D1&D4 | 21.8 | 27.9 | 1.10 ± 0.21 | 42.7* |
| CTX | 30 | 8 | I.P., D1-7 | 21.8 | 26.8 | 0.60 ± 0.18 | 68.8* |

*P < 0.01 as compared to the vehicle group

### Example 3. Compound of Formula (I) Inhibited Colon Cancer Growth in Mice

BALB/cA-nude mice, 5-6 weeks old, purchased from Shanghai SLAC Laboratory Animal Co., Ltd, were subcutaneously inoculated with Ls-174t human colon cancer cells (from ATCC). After the tumors grew to the size of 100-150 mm³, the tumor-bearing mice were randomly assigned to five groups on Day 0, and administered with physiological saline, Compound A at different doses/dosing routes, and pemetrexed disodium (Eli Lilly and Company), respectively. The detailed dosing regimen was listed in Table 4.

Mice body weights and tumor sizes were measured 2 to 3 times a week. The tumor volume (V) was calculated as (length× width²)/2. T/C (%) was calculated as (T-T₀)/(C-C₀)×100, wherein T and C represented the tumor volumes in the administration group and the vehicle group respectively at the end of the experiment, while T₀ and C₀ represented the tumor volumes in the administration group and the vehicle group respectively at the beginning of the experiment. And RTV was calculated as (Tumor volume at Day N/Tumor volume at Day 0). Mice were sacrificed on Day-14.

TABLE 4
Dosing regimen and anti-tumor effect

| | | | | Body weight | | | | | |
|---|---|---|---|---|---|---|---|---|---|
| | | | | (without | | Tumor volume | | | |
| | Dose | | Dosing | tumor) (g) | | (mm³) | | RTV at | T/C |
| Group | (mg/kg) | N | Regimen | D0 | D14 | D0 | D14 | D14 | (%) |
| Vehicle | n.a. | 12 | P.O., D0, | 24.6 | 21.3 | 133 ± 25 | 2680 ± 641 | 21.09 ± 7.19 | |
| | | | 2, 4, 8, 10; | | | | | | |
| | | | I.P., D0-4, | | | | | | |
| | | | 7-11 | | | | | | |
| Compound | 25 | 6 | P.O., D0, | 23.7 | 17.7 | 145 ± 19 | 1997 ± 316 | 13.91 ± 2.39 | 66 |
| A | | | 2, 4, 8, 10 | | | | | | |
| | 50 | 6 | P.O., D0, | 23.3 | 16.9 | 149 ± 29 | 1497 ± 282 | 10.25 ± 2.39 | 48.6* |
| | | | 4, 8 | | | | | | |
| | 50 | 6 | P.O., D0, | 23.4 | 16.2 | 147 ± 22 | 1176 ± 450 | 8.21 ± 3.55 | 38.9* |
| | | | 3, 6, 9 | | | | | | |
| Pemetrexed | 153 | 6 | I.P., D0-4, | 24.0 | 15.3 | 138 ± 33 | 1209 ± 244 | 9.14 ± 2.86 | 43.3* |
| disodium | | | 7-11 | | | | | | |

*P < 0.01 as compared to the vehicle group

No mice died of the tumor or drug administration. Both Compound A and pemetrexed disodium were capable of inhibiting tumor growth to some extent, and oral administration of Compound A at the dose of 50 mg/kg on Day 0, 4 and 8 had the best tumor growth inhibition effect.

### Example 4. Compound of Formula (I) Inhibited Non-Small Cell Lung Cancer Growth in Mice

BALB/cA-nude mice, 5-6 weeks old, purchased from Shanghai SLAC Laboratory Animal Co., Ltd, were subcutaneously inoculated with A549 human non-small cell lung cancer cells (from ATCC). After the tumors grew to the size of 100-150 mm³, the tumor-bearing mice were randomly assigned to five groups on Day 0, and administered with physiological saline, compound A at three different doses, and cisplatin (Hengrui medicine), respectively. The detailed dosing regimen was listed in Table 5 below.

Mice body weights and tumor sizes were measured 2 to 3 times a week. The tumor volume, T/C and RTV were calculated as described above. Mice were sacrificed on Day 18.

TABLE 5
Dosing regimen and anti-tumor effect

| | | | | Body | | | | | |
|---|---|---|---|---|---|---|---|---|---|
| | | | | weight | | | | | |
| | Dose | | | (with | | Tumor volume | | | |
| | (mg/ | | Dosing | tumor) (g) | | (mm³) | | | T/C |
| Group | kg) | N | Regimen | D0 | D18 | D0 | D18 | RTV at D18 | (%) |
| Vehicle | n.a. | 12 | P.O., D0, | 16.0 | 15.5 | 117 ± 24 | 2952 ± 1263 | 26.36 ± 14.1 | |
| | | | 3, 6, 9; | | | | | | |
| | | | I.P., D0, | | | | | | |
| | | | 3 | | | | | | |
| Compound | 15 | 6 | P.O., D0, | 15.6 | 15.9 | 118 ± 21 | 2135 ± 1279 | 18.67 ± 12.89 | 70.8 |
| A | | | 3, 6, 9 | | | | | | |
| | 30 | 6 | P.O., D0, | 16.0 | 16.1 | 113 ± 19 | 1449 ± 608 | 12.58 ± 4.38 | 47.7* |
| | | | 3, 6, 9 | | | | | | |
| | 60 | 6 | P.O., D0, | 16.2 | 14.7 | 110 ± 11 | 1039 ± 669 | 9.15 ± 5.33 | 34.7* |
| | | | 3, 6, 9 | | | | | | |
| Cisplatin | 5 | 6 | I.P., D0, | 16.7 | 15.3 | 110 ± 12 | 944 ± 536 | 8.89 ± 5.39 | 33.7* |
| | | | 3 | | | | | | |

*P < 0.01 as compared to the vehicle group

No mice died of the tumor or drug administration. Both Compound A and cisplatin were capable of inhibiting tumor growth to some extent, and oral administration of Compound A at the dose of 30 mg/kg on Day 0, 3, 6 and 9 had the best tumor growth inhibition effect.

### Example 5. Compound of Formula (I) Inhibited Gastric Cancer Growth in Mice

BALB/cA-nude mice, 5-6 weeks old, purchased from Shanghai SLAC Laboratory Animal Co., Ltd, were subcutaneously inoculated with SGC-7901 human gastric cancer cells (from Cell Bank, Chinese Academy of Sciences). After the tumors grew to the size of 100-150 mm³, the tumor-bearing mice were randomly assigned to five groups on Day 0, and administered with physiological saline, Compound A at three different doses, and Cisplatin (Hengrui medicine), respectively. The detailed dosing regimen was listed in Table 6 below.

Mice body weights and tumor sizes were measured 2 to 3 times a week. The tumor volume, T/C and RTV were calculated as described above. Mice were sacrificed on Day 18.

TABLE 6
Dosing regimen and anti-tumor effect

| | | | | Body weight | | | | | |
|---|---|---|---|---|---|---|---|---|---|
| | Dose | | | (with tumor) | | | | | |
| | (mg/ | | Dosing | (g) | | Tumor volume (mm³) | | RTV at | T/C |
| Group | kg) | N | Regimen | D0 | D18 | D0 | D18 | D18 | (%) |
| Vehicle | | 12 | P.O., D0, | 18.2 | 17.9 | 144 ± 58 | 1038 ± 430 | 8.7 ± 4.72 | |
| | | | 3, 6, 9, 12; | | | | | | |
| | | | I.P., D0, 6 | | | | | | |
| Compound | 15 | 6 | P.O., D0, | 18.0 | 16.7 | 162 ± 32 | 845 ± 349 | 5.57 ± 2.74 | 68.2 |
| | | | 3, 6, 9, 12 | | | | | | |
| A | 30 | 6 | P.O., D0, | 18.4 | 17.1 | 197 ± 45 | 701 ± 142 | 3.73 ± 1.16 | 45.7* |
| | | | 3, 6, 9, 12 | | | | | | |
| | 60 | 6 | P.O., D0, | 18.2 | 15.1 | 215 ± 49 | 545 ± 233 | 2.58 ± 1.07 | 31.6* |
| | | | 3, 6, 9, 12 | | | | | | |
| Cisplatin | 5 | 6 | I.P., D0, 6 | 18.4 | 16.0 | 127 ± 24 | 366 ± 201 | 2.85 ± 1.52 | 34.9* |

*P < 0.01 as compared to the vehicle group

No mice died of the tumor or drug administration. Both Compound A and cisplatin were capable of inhibiting tumor growth to some extent, and the oral administration of Compound A at the dose of 30 mg/kg on Day 0, 3, 6, 9 and 12 gave the best tumor growth inhibition effect.

### Example 6. Compound of Formula (I) Alone or in Combination with Gemcitabine Inhibited Colon Cancer Growth in Mice

F1 mice, 18-22 g, purchased from Shanghai Bi-Kai Laboratory Animal Co., Ltd, were subcutaneously inoculated with Colon26 murine colon cancer cells (from Cell Bank, Chinese Academy of Sciences) at the right axilla. After the tumors grew to the size of 100-150 mm³, the tumor-bearing mice were randomly assigned to seven groups on Day 1, and administered with physiological saline, Compound A alone or with gemcitabine, gemcitabine (Hansoh Pharma), and CTX (Hengrui Medicine), respectively. The detailed dosing regimen was listed in Table 7 below. On Day 8, the mice were weighed and sacrificed, and the tumors were collected and weighed. Tumor growth inhibition (TGI) was calculated.

No mice died of the tumor or drug administration during the test. It can be seen from Table 7 that i) Compound A alone at the dose of 60 mg/kg had comparable tumor inhibition effect to gemcitabine at the dose of 20 mg/kg and CTX at the dose of 30 mg/kg; ii) when the dose of Compound A or gemcitabine became lower, the TGI decreased accordingly; and iii) the combined use of Compound A and gemcitabine provided increased TGI compared to the monotherapies.

TABLE 7
Dosing regimen and anti-tumor effect

| | Dose | | | Body weight (without | Tumor | TGI |
|---|---|---|---|---|---|---|
| Group | (mg/kg) | N | Dosing Regimen | tumor) at D8 (g) | weight (g) | (%) |
| Compound A | 60 | 8 | P.O., D1-7 | 15.96 | 0.49 | 76.12 |
| | 30 | 8 | P.O., D1-7 | 21.57 | 1.39 | 31.88 |
| Gemcitabine | 20 | 8 | IV, D1 | 22.35 | 0.38 | 81.53 |
| | 10 | 8 | I.V., D1 | 22.31 | 0.86 | 57.51 |
| Compound A + | 30 + | 8 | P.O., D1-7; | 21.57 | 0.49 | 75.77 |
| Gemcitabine | 10 | | I.V., D1 | | | |
| CTX | 30 | 8 | I.V., D1-7 | 21.22 | 0.43 | 79.00 |
| Vehicle | n.a. | 8 | P.O., D1-7; I.V., | 21.30 | 2.03 | |
| | | | D1 | | | |

### Example 7. Compound of Formula (I) Inhibited AML Growth/Development in Mice

BALB-cA nude mice, 6 weeks old, purchased from Shanghai SLAC Laboratory Animal Co., Ltd, were subcutaneously inoculated with MV-4-11 cells (human biphenotypic B myelomonocytic leukemia cell line, from ATCC). After the tumors grew to the size of 60-150 mm³, the tumor-bearing mice were randomly assigned to five groups on Day 0, and administered with physiological saline, Compound A at two doses, and CAL101 (a PI3K δ inhibitor, APE×BIO) at two doses, respectively. The detailed dosing regimen was listed in Table 8 below. Mice body weights and tumor sizes were measured twice a week. The tumor volume, T/C and TGI (Tumor growth inhibition=(average tumor volume in vehicle group-average tumor volume in administration group)/average tumor volume in vehicle group ×100%) were calculated. Mice were sacrificed on Day 21.

TABLE 8
Dosing regimen and anti-tumor effect

| | | | | | | | | P value | |
|---|---|---|---|---|---|---|---|---|---|
| | Dose | | | | | T/C | TGI | compared | |
| | (mg/ | | Dosing | Tumor volume (mm³) | | (%) | (%) | to vehicle | Complete |
| Group | kg) | N | Regimen | D0 | D21 | D21 | D21 | group | remission |
| Vehicle | n.a. | 12 | P.O., | 78.3 ± 1.7 | 797.3 ± 85.6 | | | | 0 |
| | | | D0-20 | | | | | | |
| Compound | 30 | 6 | P.O., | 77.6 ± 3.4 | 279.2 ± 71.8 | 28 | 72 | 0.001 | 0 |
| | | | D0-20 | | | | | | |
| A | 60 | 6 | P.O., | 79.3 ± 2.6 | 76.6 ± 46.2 | 0 | 100 | 0.000 | 3 |
| | | | D0-20 | | | | | | |
| CAL 101 | 90 | 6 | P.O., | 79.6 ± 3.1 | 442.2 ± 93.8 | 50 | 50 | 0.021 | 0 |
| | | | D0-20 | | | | | | |
| | 180 | 6 | P.O., | 78.9 ± 1.9 | 361.7 ± 93.8 | 39 | 61 | 0.006 | 0 |
| | | | D0-20 | | | | | | |

No mice died of tumor or drug administration. The TGI in the high dose compound A group was as high as 100%, 3 out of 6 mice got complete remission. And it was quite obvious that the inhibitory effect of Compound A on AML was superior to that of the newly FDA approved CAL101.

The above data suggested that Compound A was capable of inhibiting sarcoma, lung tumor, gastric tumor and colon tumor growth, with the TGI in the 60-80% range, and it was highly efficacious in AML treatment, with TGI being 100% and half of the animals getting complete remission.

### Example 8. Compound of Formula (I) Alone or in Combination with Daunorubicin Inhibited AML Growth/Development in Mice

Female BALB-cA nude mice, 6-7 weeks old, purchased from Shanghai Ling-chang Biotech Co., Ltd, were subcutaneously inoculated with MV-4-11 cells (from ATCC). After the tumors grew to the size of 100-150 mm³, the tumor-bearing mice were randomly assigned to five groups on Day 0, and administered with physiological saline, Compound A, daunorubicin (HayZen), and compound A plus daunorubicin at different doses, respectively. The detailed dosing regimen was listed in Table 9 below. Mice body weights and tumor sizes were measured twice a week. The tumor volume, T/C and tumor volume based TGI were calculated as described above. Mice were sacrificed on Day 11.

TABLE 9
Dosing regimen and anti-tumor effect

| | | | | Tumor volume | | |
|---|---|---|---|---|---|---|
| Group | N | Dose (mg/kg) | Dosing Regimen | (D0/D11) | T/C (%) | TGI (%) |
| Vehicle | 12 | n.a. | P.O. + I.V., D0-10 | 151.2/2852.5 | — | — |
| Compound A + | 8 | 15 + 2 | P.O. + I.V., D0-10 | 152.8/897.7 | 28 | 72 |
| Daunorubicin | 8 | 30 + 2 | P.O. + I.V., D0-10 | 153.5/781.5 | 23 | 77 |
| Compound A | 8 | 30 | P.O., D0-10 | 150.9/2076.3 | 71 | 29 |
| Daunorubicin | 8 | 2 | I.V., D0-10 | 152.9/1167.3 | 38 | 62 |

The results were shown in Table 9. No mice died of tumor or drug administration, or had significant body weight loss. And the combined use of Compound A and daunorubicin provided better anti-tumor effect as compared to the monotherapies.

### Example 9. Compound of Formula (I) Alone or in Combination with PARP Inhibitor Induced Tumor Cell Death In Vitro

The in vitro inhibitory effect of Compound A alone or in combination with a PARP inhibitor on tumor cell growth was tested using human small cell lung cancer (SCLC) cell lines, including H69 (ATCC® HTB-119™), H526 (ATCC® CRL-5811™) and H446 (TCHu196, National Collection of Authenticated Cell Cultures).

Briefly, the cells were seeded into a 96-well dish with RPMI-1640 (10-040-CV, Coming cellgro) containing 10% fetal bovine serum (10270-106, GIBCO), 1% penicillin and 1% streptomycin (BaSalMedia, S110JV), at an initial density of 3×10³ cells per well (3×10³ cells per ml), and cultured at a 37° C., 5% CO₂ humidified incubator for 24 hours. Then, the culture media were added with Compound A (final concentration at 20 nM), olaparib (APEXBIO, A4154, a PARP inhibitor, final concentration at 10 μM), Compound A + olaparib (final concentrations at20 nM and 10 μM), or PBS (vehicle), respectively, and the cells were cultured for 48 hours. Then, cell viability was determined by MTT. The test was done in triplicate.

Cell death rate was calculated by the following formula.

Cell death rate=[1−(ODₜₑₛₜ-OD_{vehicle})]×100%

Cell death rates were analyzed using student's t-test, and group differences were deemed statistically significant when the p-value was lower than 0.05. Zheng-Jun Jin's Q value was calculated using the formula Q=E_{A+B}/(E_{A}+E_{B}-E_{A}×E_{B}) to assess the combined effect of Compound A and the PARP inhibitor, wherein E_{A+B}, E_{A}, and E_{B} referred to the cell death rates caused by the combined treatment, Compound A treatment and PARP inhibitor treatment, respectively. A Q value higher than 1.15 meant a synergistic or additive effect.

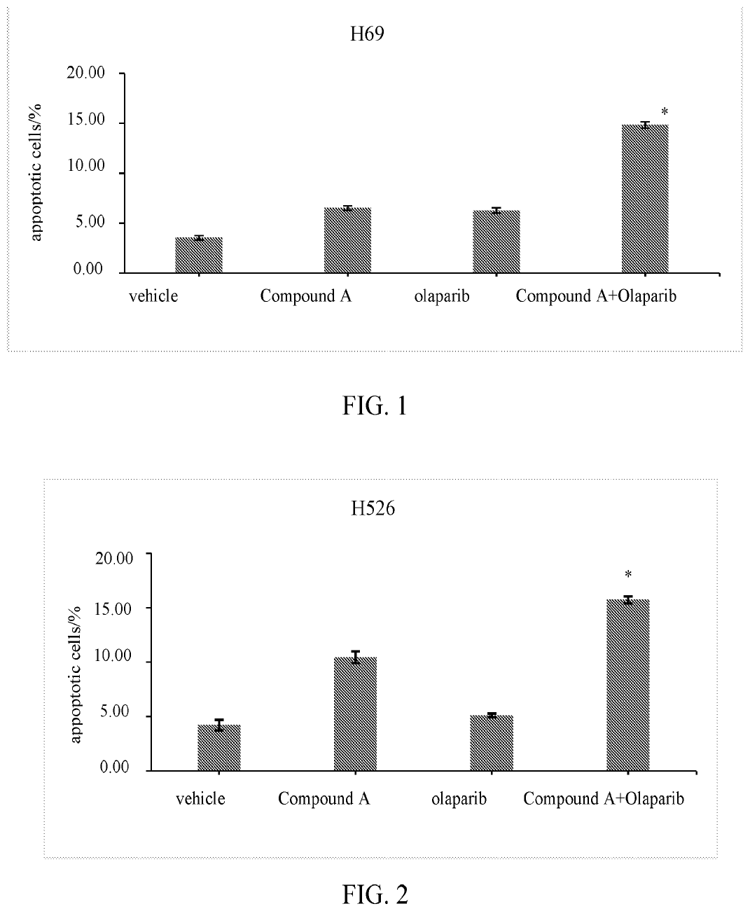

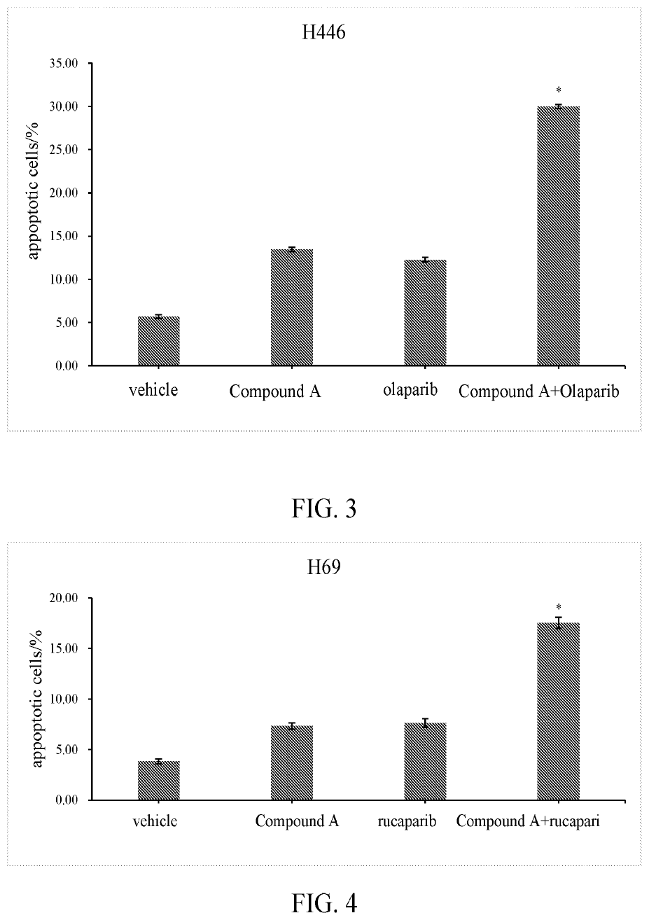

The cell death rates were shown in Table 10 and FIGS. 1 to 3.

TABLE 10
Cell death rates in different groups

| | | Final | Cell Death Rate/% | | | | Zheng-Jun |
|---|---|---|---|---|---|---|---|
| Cell line | Test articles | concentration | 1 | 2 | 3 | mean | Jin's Q value |
| H69 | Vehicle | n.a. | 3.5 | 3.34 | 3.76 | 3.53 | |
| | Compound A | 20 nM | 6.75 | 6.48 | 6.31 | 6.51 | |
| | Olaparib | 10 μM | 6 | 6.25 | 6.54 | 6.26 | |
| | Compound A + | 20 nM + 10 μM | 14.75 | 14.57 | 15.19 | 14.84 | 1.20 |
| | Olaparib | | | | | | |
| H526 | Vehicle | n.a. | 4 | 4.77 | 3.86 | 4.21 | |
| | Compound A | 20 nM | 10.5 | 9.87 | 10.95 | 10.44 | |
| | Olaparib | 10 μM | 5.25 | 4.92 | 5.13 | 5.10 | |
| | Compound A + | 20 nM + 10 μM | 15.75 | 15.39 | 16.04 | 15.73 | 1.05 |
| | Olaparib | | | | | | |
| H446 | Vehicle | n.a. | 5.73 | 5.45 | 5.9 | 5.69 | |
| | Compound A | 20 nM | 13.5 | 13.18 | 13.67 | 13.45 | |
| | Olaparib | 10 μM | 12.01 | 12.55 | 12.24 | 12.27 | |
| | Compound A + | 20 nM + 10 μM | 30 | 30.22 | 29.76 | 29.99 | 1.24 |
| | Olaparib | | | | | | |

It can be seen that the cell death rate in the combination group was significantly higher than other groups, no matter which cell line was tested, and Compound A worked, or tended to work with the PARP inhibitor in a synergistic manner to induce tumor cell death.

### Example 10. Compound of Formula (I) Alone or in Combination with PARP Inhibitor Induced Tumor Cell Death In Vitro

Another PARP inhibitor, rucaparib (APEXBIO, A4156), was tested alone or with Compound A for inhibitory effect on tumor cell growth, following the protocol of Example 9.

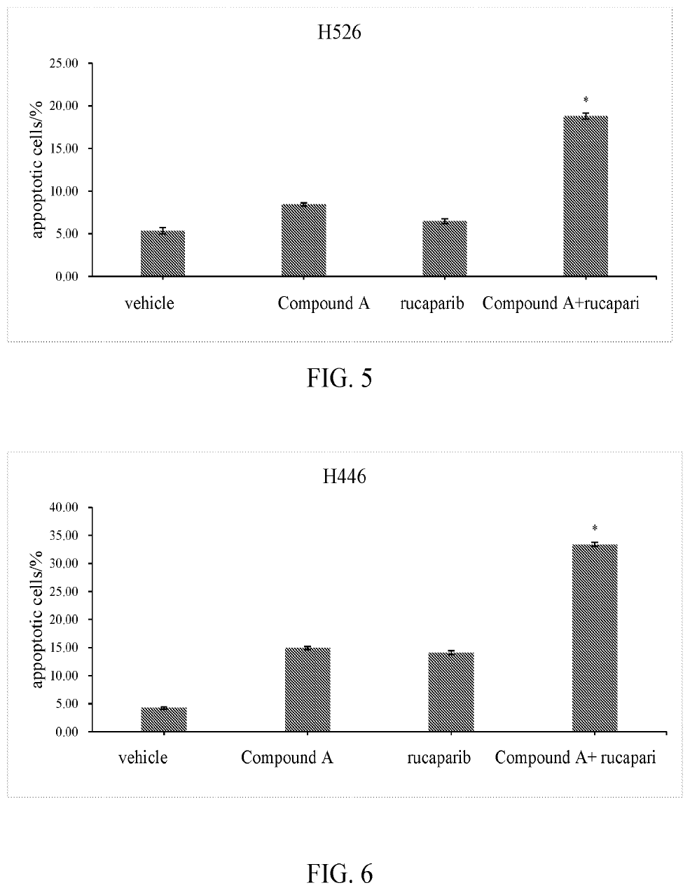

The results were shown in Table 11 and FIGS. 4 to 6.

TABLE 11
Cell death rates in different groups

| | | Final | Cell Death Rate/% | | | | Zheng-Jun |
|---|---|---|---|---|---|---|---|
| Cell line | Test articles | concentration | 1 | 2 | 3 | mean | Jin's Q value |
| H69 | Vehicle | n.a. | 3.8 | 4.08 | 3.61 | 3.83 | |
| | Compound A | 20 nM | 7.36 | 7.65 | 7.02 | 7.34 | |
| | Rucaparib | 10 μM | 7.51 | 7.3 | 8.11 | 7.64 | |
| | Compound A + | 20 nM + 10 μM | 17.68 | 16.92 | 17.99 | 17.53 | 1.22 |
| | Rucaparib | | | | | | |
| H526 | Vehicle | n.a. | 5.33 | 5.74 | 5 | 5.36 | |
| | Compound A | 20 nM | 8.29 | 8.39 | 8.64 | 8.44 | |
| | Rucaparib | 10 μM | 6.73 | 6.57 | 6.19 | 6.50 | |
| | Compound A + | 20 nM + 10 μM | 18.82 | 18.46 | 19.13 | 18.80 | 1.31 |
| | Rucaparib | | | | | | |
| H446 | Vehicle | n.a. | 4.45 | 4.28 | 4.06 | 4.26 | |
| | Compound A | 20 nM | 15 | 14.62 | 15.17 | 14.93 | |
| | Rucaparib | 10 μM | 14 | 13.79 | 14.48 | 14.09 | |
| | Compound A + | 20 nM + 10 μM | 33 | 33.54 | 33.66 | 33.40 | 1.24 |
| | Rucaparib | | | | | | |

It can be seen that the cell death rate in the combination group was significantly higher than other groups, no matter which cell line was tested, and Compound A worked with the PARP inhibitor in a synergistic manner to induce tumor cell death.

1. A method for treating leukemia in a subject in need thereof, comprising administering the subject an therapeutically effective amount of a compound of formula (I) or a pharmaceutically acceptable salt or solvent thereof, 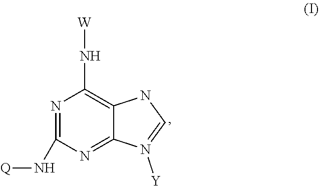
wherein W is hydrogen, an optionally substituted C₁₋₆ alkyl, an optionally substituted C₃₋₆ cycloalkyl, or an optionally substituted C₁₋₆ haloalkyl,
Y is hydrogen, or a saccharide, and
Q is hydrogen, or one selected form the group consisting of: 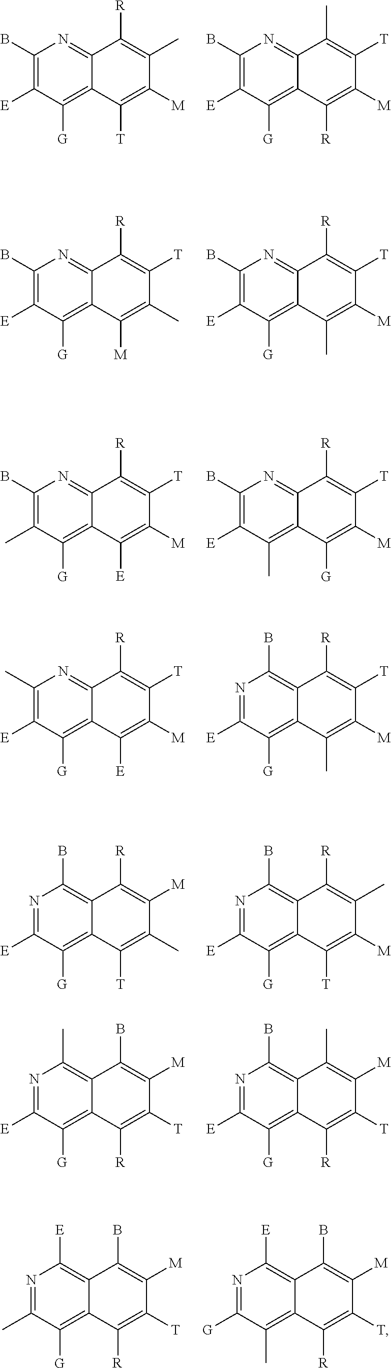
wherein B, E, G, R, T and M are independently hydrogen, an C₁₋₆ alkyl, an C₃₋₆ cycloalkyl, a halogen, a cyano, or an amino group.

2. The method according to claim 1, wherein W is selected from the group consisting of:

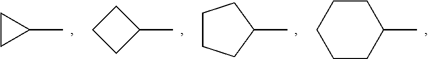

3. The method according to claim 1, wherein Q is selected from the group consisting of:

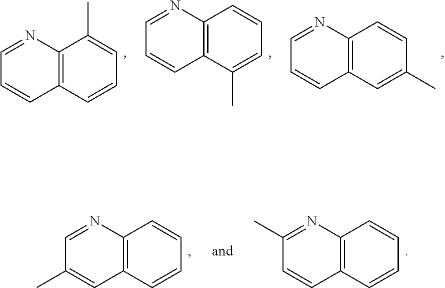

4. The method according to claim 1, wherein Y is a saccharide selected from the group consisting of: 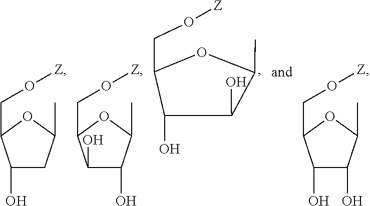
wherein Z is hydrogen or one selected from the group consisting of:

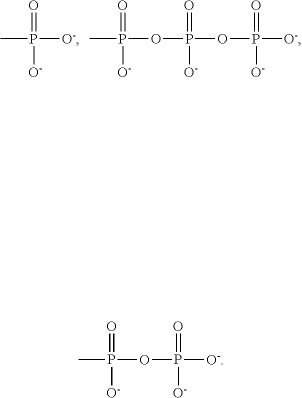

5. The method according to claim 3, wherein W is

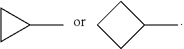

6. The method according to claim 5, wherein Q is

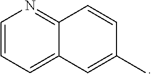

7. The method according to claim 1, wherein the compound of formula (I) is selected from the group consisting of

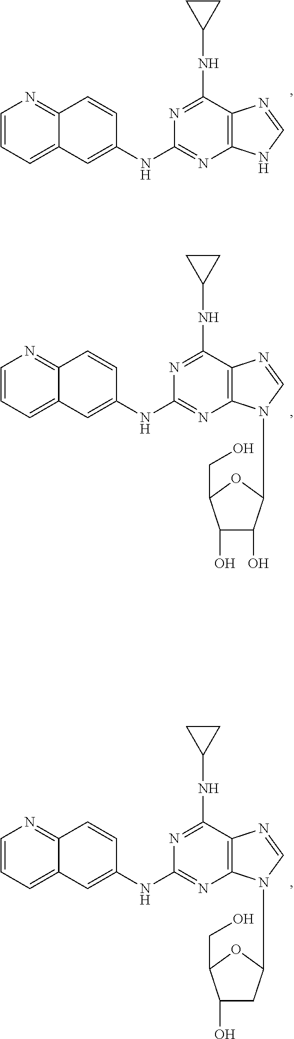 [SMILES: c1cnc2ccc(Nc3nc(NC4CC4)c4nc[nH]c4n3)cc2c1 ; OCC1OC(n2cnc3c(NC4CC4)nc(Nc4ccc5ncccc5c4)nc32)C(O)C1O ; OCC1OC(n2cnc3c(NC4CC4)nc(Nc4ccc5ncccc5c4)nc32)CC1O]

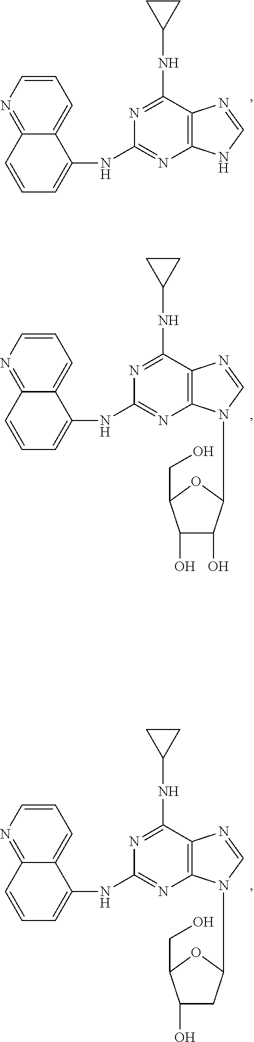 [SMILES: c1cc(Nc2nc(NC3CC3)c3nc[nH]c3n2)c2cccnc2c1 ; OCC1OC(n2cnc3c(NC4CC4)nc(Nc4cccc5ncccc45)nc32)C(O)C1O ; OCC1OC(n2cnc3c(NC4CC4)nc(Nc4cccc5ncccc45)nc32)CC1O]

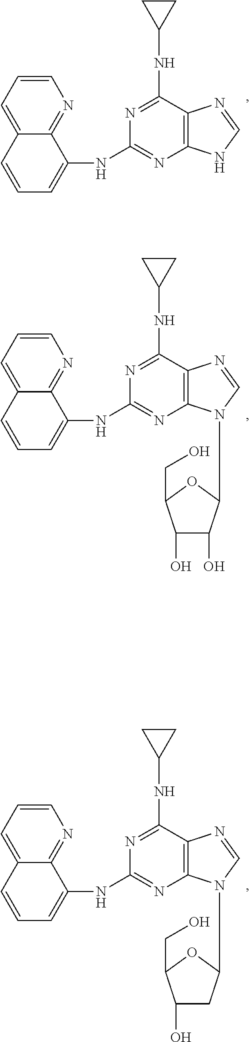 [SMILES: c1cnc2c(Nc3nc(NC4CC4)c4nc[nH]c4n3)cccc2c1 ; OCC1OC(n2cnc3c(NC4CC4)nc(Nc4cccc5cccnc45)nc32)C(O)C1O ; OCC1OC(n2cnc3c(NC4CC4)nc(Nc4cccc5cccnc45)nc32)CC1O]

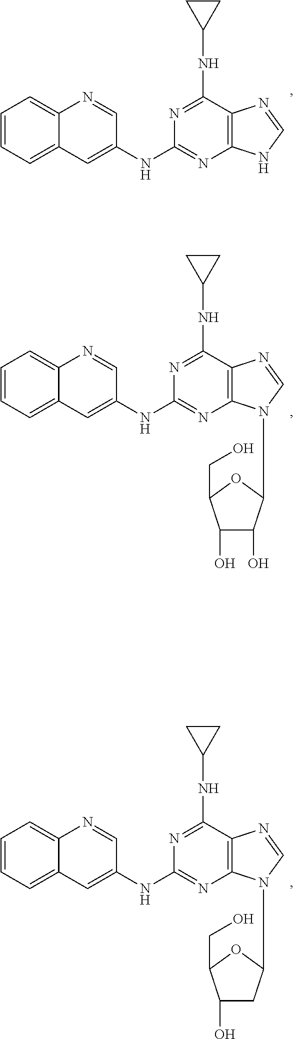 [SMILES: c1ccc2ncc(Nc3nc(NC4CC4)c4nc[nH]c4n3)cc2c1 ; OCC1OC(n2cnc3c(NC4CC4)nc(Nc4cnc5ccccc5c4)nc32)C(O)C1O ; OCC1OC(n2cnc3c(NC4CC4)nc(Nc4cnc5ccccc5c4)nc32)CC1O]

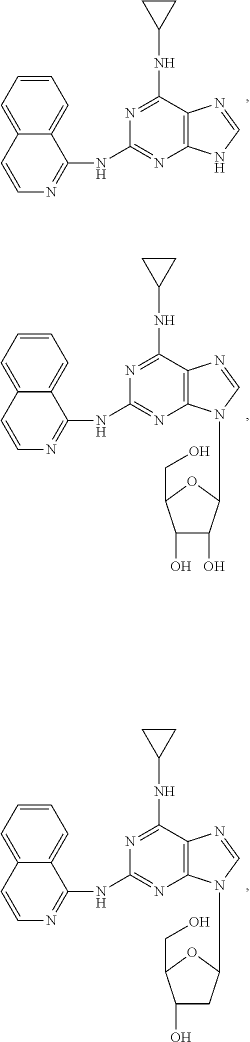 [SMILES: c1ccc2c(Nc3nc(NC4CC4)c4nc[nH]c4n3)nccc2c1 ; OCC1OC(n2cnc3c(NC4CC4)nc(Nc4nccc5ccccc45)nc32)C(O)C1O ; OCC1OC(n2cnc3c(NC4CC4)nc(Nc4nccc5ccccc45)nc32)CC1O]

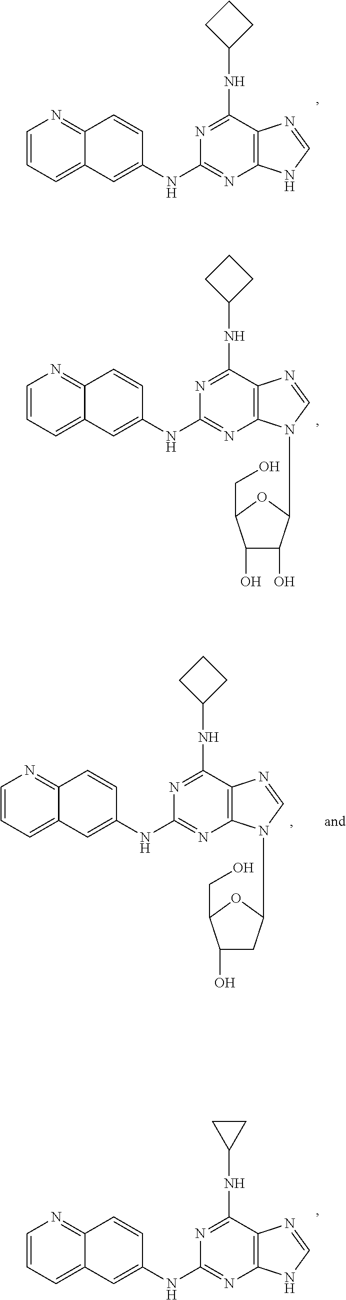 [SMILES: c1cnc2ccc(Nc3nc(NC4CCC4)c4nc[nH]c4n3)cc2c1 ; OCC1OC(n2cnc3c(NC4CCC4)nc(Nc4ccc5ncccc5c4)nc32)C(O)C1O ; OCC1OC(n2cnc3c(NC4CCC4)nc(Nc4ccc5ncccc5c4)nc32)CC1O ; c1cnc2ccc(Nc3nc(NC4CC4)c4nc[nH]c4n3)cc2c1]

8. The method according to claim 7, wherein the compound is

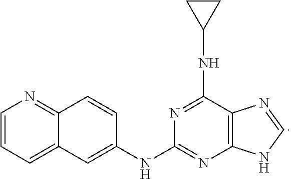 [SMILES: c1cnc2ccc(Nc3nc(NC4CC4)c4nc[nH]c4n3)cc2c1]

9. The method according to claim 1, wherein the leukemia is acute leukemia, chronic leukemia, or acute myeloid leukemia.

10. A method for treating a cancer in a subject in need thereof, comprising administering a therapeutically effective amount of a compound of formula (I), or a pharmaceutically acceptable salt or solvent thereof, in combination with a poly(ADP)-ribose polymerase (PARP) inhibitor and/or a chemotherapeutic agent, 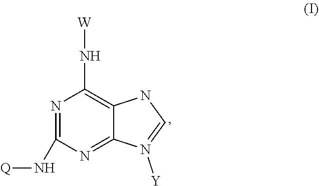
wherein W is hydrogen, an optionally substituted C₁₋₆ alkyl, an optionally substituted C₃₋₆ cycloalkyl, or an optionally substituted C₁₋₆ haloalkyl,
Y is hydrogen, or a saccharide, and
Q is hydrogen, or one selected form the group consisting of: 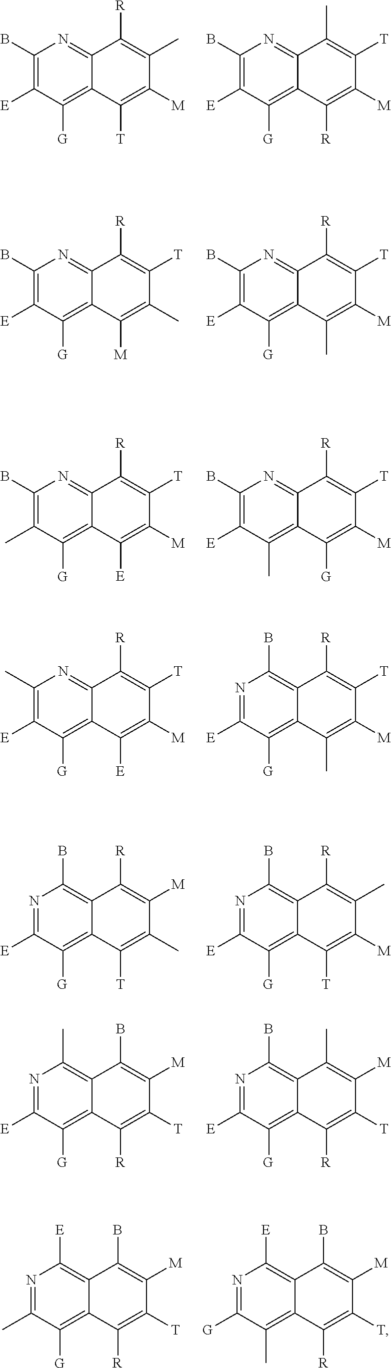
wherein B, E, G, R, T and M are independently hydrogen, an C₁₋₆ alkyl, an C₃₋₆ cycloalkyl, a halogen, a cyano, or an amino group.

11. The method according to claim 10, wherein W is selected from the group consisting of:

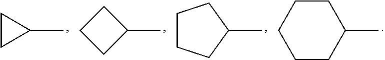

12. The method according to claim 10, wherein Q is selected from the group consisting of:

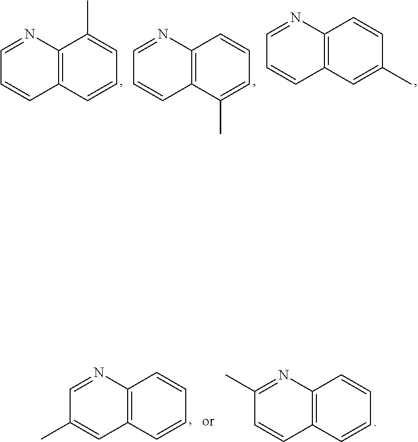

13. The method according to claim 10, wherein Y is a saccharide selected from the group consisting of: 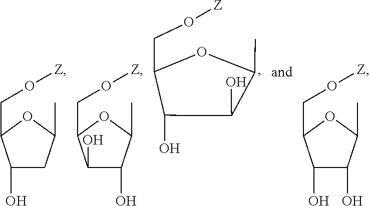
wherein Z is hydrogen or one selected from the group consisting of:

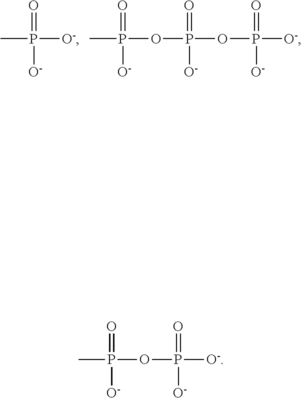

14. The method according to claim 12, wherein W

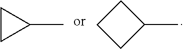

15. The method according to claim 14, wherein Q is

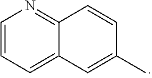

16. The method according to claim 10, wherein the compound of formula (I) is selected from the group consisting of

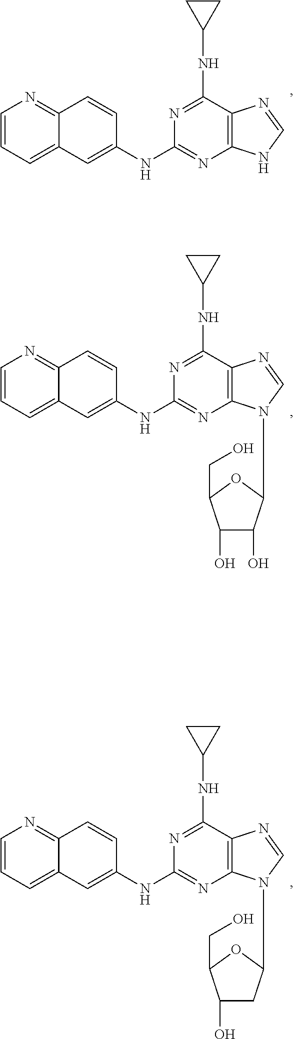 [SMILES: c1cnc2ccc(Nc3nc(NC4CC4)c4nc[nH]c4n3)cc2c1 ; OCC1OC(n2cnc3c(NC4CC4)nc(Nc4ccc5ncccc5c4)nc32)C(O)C1O ; OCC1OC(n2cnc3c(NC4CC4)nc(Nc4ccc5ncccc5c4)nc32)CC1O]

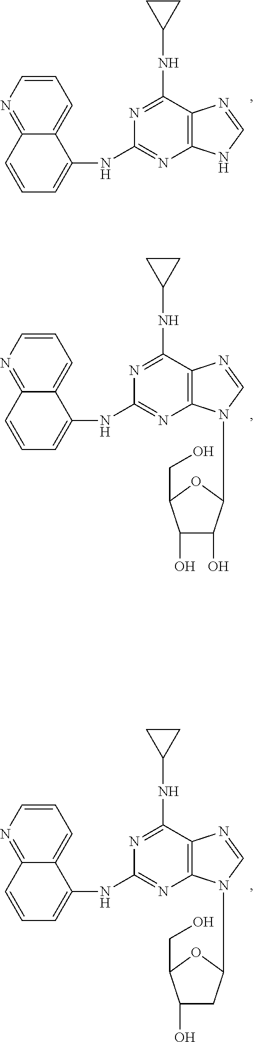 [SMILES: c1cc(Nc2nc(NC3CC3)c3nc[nH]c3n2)c2cccnc2c1 ; OCC1OC(n2cnc3c(NC4CC4)nc(Nc4cccc5ncccc45)nc32)C(O)C1O ; OCC1OC(n2cnc3c(NC4CC4)nc(Nc4cccc5ncccc45)nc32)CC1O]

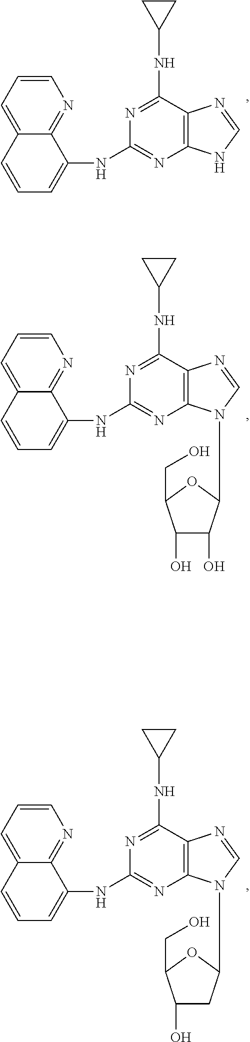 [SMILES: c1cnc2c(Nc3nc(NC4CC4)c4nc[nH]c4n3)cccc2c1 ; OCC1OC(n2cnc3c(NC4CC4)nc(Nc4cccc5cccnc45)nc32)C(O)C1O ; OCC1OC(n2cnc3c(NC4CC4)nc(Nc4cccc5cccnc45)nc32)CC1O]

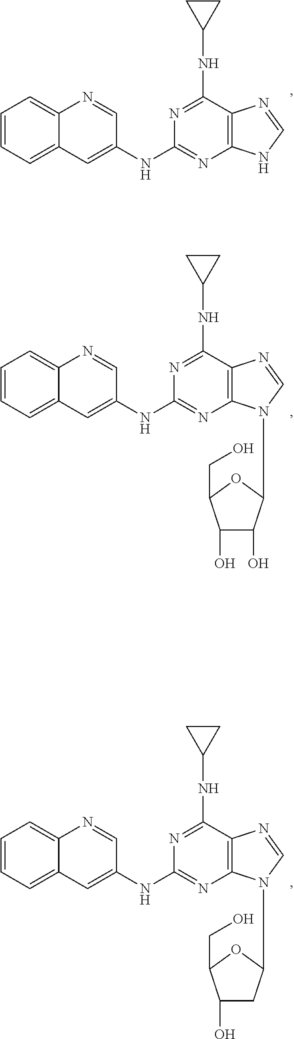 [SMILES: c1ccc2ncc(Nc3nc(NC4CC4)c4nc[nH]c4n3)cc2c1 ; OCC1OC(n2cnc3c(NC4CC4)nc(Nc4cnc5ccccc5c4)nc32)C(O)C1O ; OCC1OC(n2cnc3c(NC4CC4)nc(Nc4cnc5ccccc5c4)nc32)CC1O]

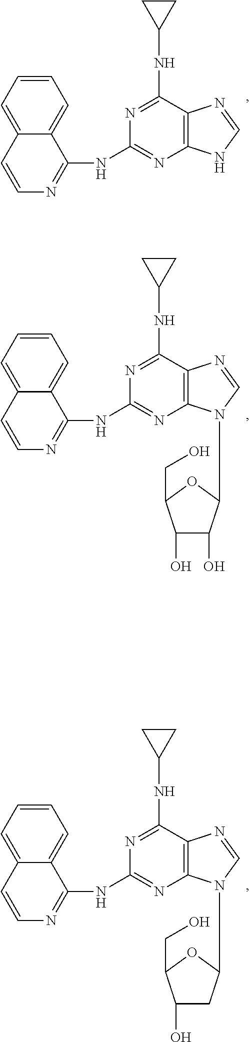 [SMILES: c1ccc2c(Nc3nc(NC4CC4)c4nc[nH]c4n3)nccc2c1 ; OCC1OC(n2cnc3c(NC4CC4)nc(Nc4nccc5ccccc45)nc32)C(O)C1O ; OCC1OC(n2cnc3c(NC4CC4)nc(Nc4nccc5ccccc45)nc32)CC1O]

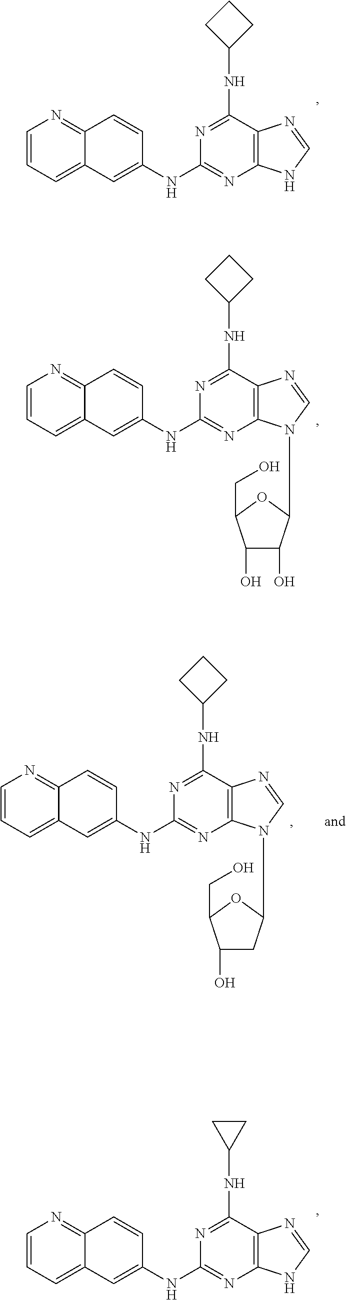 [SMILES: c1cnc2ccc(Nc3nc(NC4CCC4)c4nc[nH]c4n3)cc2c1 ; OCC1OC(n2cnc3c(NC4CCC4)nc(Nc4ccc5ncccc5c4)nc32)C(O)C1O ; OCC1OC(n2cnc3c(NC4CCC4)nc(Nc4ccc5ncccc5c4)nc32)CC1O ; c1cnc2ccc(Nc3nc(NC4CC4)c4nc[nH]c4n3)cc2c1]

17. The method according to claim 16, wherein the compound is

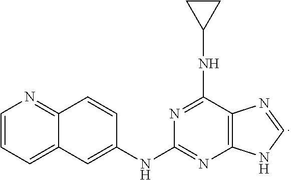 [SMILES: c1cnc2ccc(Nc3nc(NC4CC4)c4nc[nH]c4n3)cc2c1]

18. The method according to claim 10, wherein the PARP inhibitor is selected from the group consisting of olaparib, rucaparib, niraparib, and talazoparib.

19. The method according to claim 10, wherein the chemotherapeutic agent is selected from the group consisting of cisplatin, pemetrexed, gemcitabine, cytarabine, hydroxycarbamide, temozolomide, irinotecan, cyclophosphamide, mitoxantrone, etoposide, folinic acid, fludarabine, and fluorouracil.

20. The method according to claim 10, wherein the cancer is a solid cancer selected from the group consisting of lung, prostate, ovarian, brain, breast, skin, bladder, colon, gastrointestinal, head and neck, gastric, pancreas, neurologic, renal, and liver cancer, or a hematological cancer selected from the group consisting of lymphocytic leukemia, myeloid leukemia, non-Hodgkin lymphoma, and Hodgkin lymphoma.
